(19) United States
(12) Patent Application Publication
(10) Pub. No.: US 2005/0153297 A1
(43) Pub. Date: Jul. 14, 2005

(54) Method for determining oligonucleotide concentration

(76) Inventors: Ateeq Ahmad, Wadsworth, IL (US); Sumsullah Khan, Gurnee, IL (US); Imran Ahmad, Wadsworth, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/515,788

(22) PCT Filed: May 29, 2003
(86) PCT No.: PCT/US03/16874

Related U.S. Application Data
(60) Provisional application No. 60/384,222, filed on May 29, 2002.

(51) Int. Cl. C12Q 1/68; C07H 21/04

(52) U.S. Cl. 435/6; 536/25.4

(57) ABSTRACT

The invention provides a simple, sensitive method to determine the concentration of oligonucleotides in biological matrices, such as plasma. The method involves obtaining a biological specimen containing a concentration of an oligonucleotide, removing an amount of protein from the sample, subjecting the sample to a chromatographic separation, and analyzing the eluant for the amount of oligonucleotide by mass spectrometry. The assay provides a reliable measure of the concentration of oligonucleotide in the concentration range of about 5 to about 10,000 ng/mL of sample.

### FIELD OF THE INVENTION

This invention pertains to methods for quantifying oligonucleotides in biological matrices.

### BACKGROUND OF THE INVENTION

The treatment of human diseases with oligonucleotides is becoming a more common therapeutic approach. There are numerous clinical trials in which oligonucleotides are being studied for therapeutic use against diseases such as cancer, human viral diseases, and inflammatory disorders. In cancer therapy for example, oligonucleotides can be used to disrupt expression of gene products for cancer-related genes such as c-raf-1. For example, antisense c-raf-1 cDNA transfection inhibits biosynthesis of Raf-1, a cytosolic protein serine/threonine kinase which is associated with delayed tumor growth. To facilitate studies of drugs, such as this, new assays are needed that can be used to quickly and reliably determine their concentration in biological samples, such as plasma.

The invention provides such a method. These and other advantages of the invention, as well as additional inventive features, will be apparent from the description of the invention provided herein.

### SUMMARY OF THE INVENTION

The invention provides a simple, sensitive method to determine the concentration of oligonucleotides in biological matrices, such as blood plasma The method involves obtaining a biological specimen containing a concentration of an oligonucleotide, removing an amount of protein from the sample, subjecting the sample to a chromatographic separation, and analyzing the eluant for the amount of oligonucleotide by mass spectrometry. The assay provides a reliable measure of the concentration of oligonucleotide in the concentration range of about 5 to about 10,000 ng/in L of sample.

In accordance with the inventive method, the amount of protein removed from the sample can be all (e.g., substantially all) or any suitable amount of the protein. Many methods of precipitation of protein are known and can be used so long as the calibration curves remain linear. For example, proteins can be removed by precipitation with organic solvents such as acetonitrile.

The chromatographic separation can be of any suitable protocol sufficient to achieve separation sufficient to permit the analysis. Preferably, the sample is subjected to high performance chromatography, more preferably high performance reverse phase chromatography. In a preferred embodiment, the eluant is analyzed by multiple reaction monitoring by electrospray ionization mass spectrometer detection.

In alternative embodiments, the method further comprises subjecting the sample to solid phase extraction to purify the oligonucleotide. The extraction can be achieved, for example, with a reverse phase chromatography material.

### DETAILED DESCRIPTION

The present invention is directed to a method for determining the concentration of an oligonucleotide in biological samples, such as blood plasma. In general, samples containing oligonucleotide can be spiked with internal standard, processed by protein precipitation, followed by solid phase extraction, and analyzed using high performance chromatography (HPLC), such as reverse phase chromatography, with Z-Spray electrospray ionization MS/MS detection. Negative ions for oligonucleotide can be monitored in multiple reaction monitoring (MRM) mode. The oligonucleotide to internal standard peak area ratios can be used to create a linear calibration curve using a suitable regression analysis, such as a 1/x² weighted least squares analysis. The method can be used to measure oligonucleotide concentrations in the range of about 5 to about 10,000 ng/mL (such as from 8 to 10,000 ng/mL) of sample.

The following definitions are used:

$$Precision = \% Coefficient of Variation ( \% CV ) = \frac{standard deviation}{mean concentration} * 100$$

$$Accuracy = \% Difference ( \% Diff ) = \frac{mean found concentration - nominal concentration}{nominal concentration} * 100$$

$$\% Recovery = \frac{mean peak area of extracted samples}{mean peak area of unextracted samples} * 100$$

The within-run and between-run precision is 2.3 to 14% and 4.5 to 12.3% respectively. The within-run and between-run accuracy is −8.8 to 7.8% and −11.4 to 3.2%, respectively.

The following examples further illustrate the invention but, of course, should not be construed as in any way limiting its scope.

### EXAMPLE 1

This example demonstrates a high performance liquid chromatography-tandem mass spectrometry (LC/MS/MS) method to determine the concentration of an oligonucleotide containing 15 nucleotide residues in a human plasma milieu. The sequence of the oligonucleotide was 5′-GTGCTCCATTGATGC-3′. This example also shows that the assay can be used to determine oligonucleotide concentrations between about 5 ng/mL to 10000 ng/mL in a biological sample.

The oligonucleotide was prepared in a liposomal formulation wherein lipids (5 mg DDAB, 20 mg phosphatidylcholine, 5 mg cholesterol and 0.3 mg a-tocopherol) were dissolved in 4 mL t-butanol, filtered through a 0.22 μ filter and lyophilized. The lyophilized lipids were reconstituted at room temperature with 2.0 mg/mL of oligonucleotide in normal saline at an oligonucleotide to lipid mass ratio of 1:15 and vortexed vigorously for 2 min. The vials were then hydrated at room temperature for 2 h. At the end of hydration, vials were sonicated for 10 min in a bath type sonicator (Model XL 2020, Model XL 2020, Misonix Inc. Farmingdale, N.Y.).

Seven non-zero standards containing 8, 20, 100, 300, 1000, 3000, and 10000 ng/mL of oligonucleotide were prepared to generate a standard curve. Five quality control human plasma samples were prepared from human sodium heparin plasma to contain a liposomal formulation of the oligonucleotide at oligonucleotide concentrations of 8, 20, 200, 2500, and 8000 ng/mL. These samples were used for assay validation parameters and stored at about −20° C. The plasma samples (1.0 mL) were treated with acetonitrile to precipitate proteins. Solid phase extraction of oligonucleotide and the internal standard were done using a Waters Oasis™ C18 cartridge by standard methods. The extracts were evaporated to dryness and reconstituted with a solution of 5 mM ammonium acetate, pH 7.5. Samples were injected onto a Synergi Max-RP (50×2 mm, 4 μm) analytical column with a solvent delivery system (LC-10Ad vp, Shimadzu Corporation), vacuum degasser (DGU-14 A, Shimadzu Corporation) and autoinjector (PE Series 200 Injector, Perkin Elmer). The analytes were eluted with a methanol/water gradient of from 10% methanol to 90% methanol in about 1 minute in the presence of ammonium acetate at pH 8.0. The chromatographic run time was 7 minutes. Micromass Quattro Ultima triple quadrupole mass spectrometer with electro spray ionization source at −25 V cone voltage and 30 eV collision energy was used to detect the analytes by multiple reaction monitoring NM in negative ion mode. The mass transitions at m/z 1146.2→745.9 for the oligonucleotide and m/z 1128.72→731.9 for internal standard were monitored. The amount of oligonucleotide was determined by determining the relative peak area ratios. The standard curve was linear between 8 and 10,000 ng/mL of oligonucleotide standard and was used to determine oligonucleotide concentrations with better than 90% accuracy.

Table 1 shows a summary of validation parameters for LC-MS/MS assay of oligonucleotide in human plasma

TABLE 1
Sample Volume: 1000 μL

| | | Within-run | Within-run | Between-run | Between-run |
|---|---|---|---|---|---|
| | Assay | Precision | Accuracy | Precision | Accuracy |
| Analyte | Range | (% CV) | (% Diff) | (% CV) | (% Diff) |
| Antisense | 8 to 10000 ng/mL | 2.3 to 14.0% | −8.8 to 7.8% | 4.5 to 12.3% | −11.4 to −3.2% |
| Oligonucleotide | | | | | |

Table 2 shows the within-run precision and accuracy of the antisense oligonucleotide in human plasma

TABLE 2

| Concentration (ng/mL) | Precision (% CV) | Accuracy (% Diff) |
|---|---|---|
| 8 | 14.0 | −5.0 |
| 20 | 10.0 | 5.2 |
| 200 | 2.3 | −8.8 |
| 2500 | 6.6 | 7.8 |
| 8000 | 3.8 | 4.6 |

Table 3 shows the between-run precision and accuracy of the antisense oligonucleotide in human plasma

TABLE 3

| Concentration (ng/mL) | Precision (% CV) | Accuracy (% Diff) |
|---|---|---|
| 20 | 12.3 | −3.5 |
| 200 | 4.5 | −11.4 |
| 2500 | 7.9 | −5.7 |
| 8000 | 5.9 | −3.2 |

Table 4 shows the specificity test of the antisense oligonucleotide in twelve ent lots of human plasma

TABLE 4

| Concentration | | |
|---|---|---|
| (ng/mL) | Precision (% CV) | Accuracy (% Diff) |
| 0 | Interference | Interference |
| | <20% of LOQ) | <20% of LOQ) |
| 8 | 13.4 | −0.8 |

Table 5 shows the room temperature bench-top stability of the antisense oligonucleotide in human plasma

TABLE 5

| Nominal | Mean found | | |
|---|---|---|---|
| concentration | concentration | | |
| (ng/mL) | (ng/mL) | Precision (% CV) | Accuracy (% Diff) |
| 200 | 208 | 2.3 | 4.3 |
| 2500 | 2174 | 3.9 | −13.1 |
| 8000 | 6911 | 3.6 | −13.6 |

Table 6 shows the 34 hour autosampler stability at room-temperature of the antisense oligonucleotide in human plasma

TABLE 6

| Nominal | Mean found | | |
|---|---|---|---|
| concentration | concentration | | |
| (ng/mL) | (ng/mL) | Precision (% CV) | Accuracy (% Diff) |
| 20 | 17.47 | 10.0 | −12.6 |
| 200 | 229 | 7.4 | 14.7 |
| 2500 | 2693 | 9.4 | 7.7 |
| 8000 | 7660 | 1.7 | −4.3 |

Table 7 shows the 51 hour autosampler stability at 4° C. of the antisense oligonucleotide in human plasma

TABLE 7

| Nominal | Mean found | | |
|---|---|---|---|
| concentration | concentration | | |
| (ng/mL) | (ng/mL) | Precision (% CV) | Accuracy (% Diff) |
| 20 | 22.05 | 12.1 | 10.3 |
| 200 | 191 | 2.8 | −4.5 |
| 2500 | 2437 | 4.3 | −2.5 |
| 8000 | 8199 | 2.4 | 2.5 |

Table 8 shows the 3 cylces freeze/thaw stability of the antisense oligonucleotide in human plasma

TABLE 8

| Nominal | Mean found | | |
|---|---|---|---|
| concentration | concentration | | |
| (ng/mL) | (ng/mL) | Precision (% CV) | Accuracy (% Diff) |
| 200 | 186 | 6.2 | −7.0 |
| 2500 | 2493 | 2.2 | −0.3 |
| 8000 | 7875 | 4.9 | −1.6 |

Table 9 shows the 61 days long-term storage stability at −20° C. of the antisense oligonucleotide in human plasma

TABLE 9

| Nominal | Day 0 mean | Day 61 mean | |
|---|---|---|---|
| concentration | concentration | concentration | |
| (ng/mL) | (ng/mL) | (ng/mL) | (% Diff) |
| 200 | 233 | 219 | −6.0 |
| 2500 | 2893 | 2532 | −12.5 |
| 8000 | 8676 | 8690 | 0.2 |

Table 10 shows that sample can be diluted with blank matrix without effecting the final concentration determination. Human plasma samples prepared at three concentrations (25, 75, and 100 μg/mL) were diluted in six replicates with pooled blank human plasma at dilution factors of 10, 100 and 1000, respectively. The results were corrected with the dilution factor and compared to the nominal concentration. The difference between the mean of the adjusted concentration (found concentration multiplied by dilution factor) and the nominal concentration of oligonucleotide was within the acceptable range as shown below in Table 10.

TABLE 10

| Nominal | | Mean found | | |
|---|---|---|---|---|
| Concentration | Dilution | concentration | Precision | Accuracy |
| (ng/mL) | factor | (ng/mL) | (% CV) | (% Diff.) |
| 25,000 | 10 | 24998 | 1.7 | 0.00 |
| 75,000 | 100 | 71290 | 2.9 | −4.9 |
| 100,000 | 1000 | 90177 | 7.9 | −9.8 |

A recovery study was carried out to evaluate the efficiency and reproducibility of the extraction process. The recovery was determined at three standard concentrations (200, 2500, and 8000 ng/mL, n=6) for antisense oligonucleotide and at one concentration (1000 ng/mL, n=6) for the internal standard. The peak areas of the reference, or unextracted samples, were determined by spiking an equivalent amount of oligonucleotide analyte into an extract of blank plasma and injecting onto the LC/MS/MS. Recovery of oligonucleotide drug and internal standard were determined from the ratio of the mean peak area of extracted samples to the mean peak area of reference samples using the equation provided in Section 9. The results in Table 11 show that the recovery is about 30% at each oligonucleotide concentration level.

TABLE 11

| Nominal | | | |
|---|---|---|---|
| Concentration | Extracted | Unextracted | |
| (ng/mL) | (Mean Peak Area) | (Mean Peak Area) | Recovery (%) |
| 200 | 12742 | 40918 | 31.1 |
| 2500 | 170833 | 548872 | 31.1 |
| 8000 | 563627 | 1621892 | 34.8 |

This example shows that the method is robust and reproducible from 8 ng/mL to 10000 ng/mL and the range may be extended up to 100,000 ng/mL by dilution. The method is free from any interference of matrix or dilution effect, and meets the sensitivity and reproducibility criteria needed for pharmacokinetic studies of oligonucleotides in human plasma.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for determining the amount of an oligonucleotide in a biological sample comprising obtaining a biological sample containing an amount of an oligonucleotide, removing an amount of protein from the sample, subjecting the sample to a chromatographic separation, and analyzing the eluant by mass spectrometry to determine the amount of oligonucleotide in the sample.

2. A method for determining the concentration of an oligonucleotide in a biological sample comprising obtaining a biological sample containing a concentration of an oligonucleotide, removing an amount of protein from the sample, subjecting the sample to a chromatographic separation, and analyzing the eluant by mass spectrometry to determine the concentration of oligonucleotide in the sample.

3. The method of claim 1, wherein protein is removed from the sample by precipitation with an organic agent.

4. The method of claim 3, wherein the organic agent is acetonitrile.

5. The method of claim 1, wherein the chromatographic separation is high performance liquid chromatography.

6. The method of claim 1, wherein the chromatographic separation is high performance reverse chromatography.

7. (canceled)

8. The method of claim 1 further comprising the step of subjecting the sample to solid phase extraction to purify the oligonucleotide.

9. The method of claim 8, wherein the solid phase extraction uses a reverse phase chromatography material.

10. The method of claim 1, wherein the eluant is analyzed by tandem mass spectrometry.

11. The method of claim 10, wherein the tandem mass spectrometry uses multiple reaction monitoring of negative anions by Z-spray electrospray ionization mass spectrometer detection.

12. The method of claim 1, wherein the oligonucleotide is quantified by tandem mass spectrometry.

13. The method of claim 12, wherein the tandem mass spectrometry uses multiple reaction monitoring of negative anions by Z-spray electrospray ionization mass spectrometer detection.

14. The method of claim 2, wherein protein is removed from the sample by precipitation with an organic agent.

15. The method of claim 14, wherein the organic agent is acetonitrile.

16. The method of claim 2, wherein the chromatographic separation is high performance liquid chromatography.

17. The method of claim 2, wherein the chromatographic separation is high performance reverse chromatography.

18. The method of claim 2 further comprising the step of subjecting the sample to solid phase extraction to purify the oligonucleotide.

19. The method of claim 18, wherein the solid phase extraction uses a reverse phase chromatography material.

20. The method of claim 2, wherein the eluant is analyzed by tandem mass spectrometry.

21. The method of claim 20, wherein the tandem mass spectrometry uses multiple reaction monitoring of negative anions by Z-spray electrospray ionization mass spectrometer detection.

22. The method of claim 2, wherein the oligonucleotide is quantified by tandem mass spectrometry.

23. The method of claim 22, wherein the tandem mass spectrometry uses multiple reaction monitoring of negative anions by Z-spray electrospray ionization mass spectrometer detection.
